(19) United States
(12) Patent Application Publication
(10) Pub. No.: US 2024/0358892 A1
(43) Pub. Date: Oct. 31, 2024

(54) INJECTABLE SHEAR-THINNING COMPOSITIONS AND USES THEREOF

(71) Applicant: Boston Scientific Scimed, Inc., Maple Grove, MN (US)

(72) Inventors: Nicolas Ball-Jones, Minneapolis, MN (US); Samuel Michael Hanson, Minneapolis, MN (US); Gary Thomas Ohrt, Corcoran, MN (US); Sasha Viola Rios, Minneapolis, MN (US); Paul Vincent Grosso, Maple Grove, MN (US)

(73) Assignee: Boston Scientific Scimed, Inc., Maple Grove, MN (US)

(21) Appl. No.: 18/647,746

(22) Filed: Apr. 26, 2024

Related U.S. Application Data
(60) Provisional application No. 63/462,503, filed on Apr. 27, 2023.

(51) Int. Cl. A61L 27/26; A61L 27/52

(52) U.S. Cl. CPC A61L 27/26; A61L 27/52; A61L 2400/06

(57) ABSTRACT

In some embodiments, the present disclosure pertains to an injectable shear-thinning composition that comprises (a) one or more types of polymers, (b) one or more types of microparticles and (c) water. In some embodiments, the present disclosure pertains to a kit that comprises (a) one or more containers that contain an injectable shear-thinning composition that comprises one or more types of polymers, one or more types of microparticles and water and (b) a delivery device. In some embodiments, the present disclosure pertains to a medical procedure comprising administering to a subject an injectable shear-thinning composition that comprises one or more types of polymers, one or more types of microparticles and water.

### CROSS-REFERENCE TO RELATED APPLICATIONS

This application claims the benefit of U.S. Provisional Patent Application Ser. No. 63/462,503 filed on Apr. 27, 2023, the disclosure of which is incorporated herein by reference.

### FIELD

The present disclosure relates to injectable shear-thinning compositions and methods of making and using injectable shear-thinning compositions. The injectable shear-thinning compositions are useful, for example, in various medical procedures.

### BACKGROUND

Injectable shear-thinning compositions are attractive due to their minimally invasive delivery procedure, providing reduced healing time, reduced scarring, decreased risk of infection, and ease of delivery compared with surgically implanted materials. Injectable shear-thinning compositions are especially useful for applications where the final form and shape are either not important or are defined by the void or space into which they are injected. Due to their ease of delivery, injectable shear-thinning compositions are potentially useful in a number of areas such as providing a structural or space-filling function, functioning as embolic agents for diverting or eliminating flow in blood vessels, acting as tissue engineering compositions, and delivery of drugs, with the injectable shear-thinning compositions providing greater drug concentration at the desired site of action while minimizing the systemic drug concentration and associated side effects.

There is an ongoing need in the biomedical arts for novel injectable shear-thinning compositions.

### SUMMARY

In some embodiments, the present disclosure pertains to an injectable shear-thinning composition that comprises (a) one or more types of polymers, (b) one or more types of microparticles and (c) water.

In some embodiments, the injectable shear-thinning composition is a physically crosslinked hydrogel composition.

In some embodiments, which can be used in conjunction with the above embodiments, the one or more types of polymers comprise a polymer having a net-negative charge and/or a polymer having a net-positive-charge.

In some embodiments, which can be used in conjunction with the above embodiments, the one or more types of polymers are selected from polysaccharides, proteins, polypeptides, and synthetic polymers.

In some embodiments, which can be used in conjunction with the above embodiments, the one or more types of polymers are selected from non-animal-derived proteins and non-animal-derived polysaccharides.

In some embodiments, which can be used in conjunction with the above embodiments, the one or more types of microparticles are selected from metallic microparticles, metal oxide microparticles, carbon based microparticles, silicate microparticles, lipid microparticles and crosslinked charged polymer microparticles.

In some embodiments, which can be used in conjunction with the above embodiments, the one or more types of microparticles comprise microparticles having a net-negative charge and/or microparticles having a net-positive charge.

In some embodiments, which can be used in conjunction with the above embodiments, the one or more types of microparticles have a size ranging from 10 nm to 50 μm in longest dimension.

In some embodiments, which can be used in conjunction with the above embodiments, the one or more types of polymers comprise a polymer having a first net charge and wherein the one or more types of microparticles comprise microparticles having a second net charge that is opposite in sign to the first net charge.

In some embodiments, which can be used in conjunction with the above embodiments, the injectable shear-thinning composition further comprises one or more additional agents selected from therapeutic agents, imaging agents, colorants, tonicity adjusting agents, suspension agents, wetting agents, and pH adjusting agents.

In some embodiments, which can be used in conjunction with the above embodiments, the injectable shear-thinning composition is a sterile composition.

In some embodiments, which can be used in conjunction with the above embodiments, the injectable shear-thinning composition is provided in a container.

In some embodiments, which can be used in conjunction with the above embodiments, the injectable shear-thinning composition is provided in a preloaded syringe.

In some embodiments, the present disclosure pertains to a kit that comprises (a) one or more containers that contain an injectable shear-thinning composition in accordance with any of the above embodiments and (b) a delivery device.

In some embodiments, the delivery device comprises a syringe, a needle and optionally, a catheter.

In some embodiments, the present disclosure pertains to a medical procedure comprising administering to a subject an injectable shear-thinning composition in accordance with any of the above embodiments.

In some embodiments, the method comprises injecting the injectable shear-thinning composition into the subject.

In some embodiments, which can be used in conjunction with the above embodiments, the administering comprises parenteral administration.

In some embodiments, which can be used in conjunction with the above embodiments, the administering is performed using a catheter or a syringe.

In some embodiments, which can be used in conjunction with the above embodiments, the administering is performed under image guidance.

Potential benefits of the present disclosure include one or more of the following, among others: reduced injection force, improved cohesivity in larger or high-flow vasculature, improved shear-thinning behavior to allow for more distal penetration when used as an embolic agent, improved therapeutic agent release profiles, and enhanced radiopacity.

### BRIEF DESCRIPTION OF THE DRAWINGS

FIG. 1 schematically illustrates a catheter and a syringe that is loaded with an injectable shear-thinning composition, in accordance with an embodiment of the present disclosure.

### DETAILED DESCRIPTION

In some aspects, the present disclosure pertains to injectable shear-thinning compositions that comprise (a) one or more types of polymers, (b) one or more types of microparticles and (c) water. A composition is shear thinning if the viscosity decreases as the shear rate increases.

In some embodiments, the injectable shear-thinning compositions of the present disclosure are physically crosslinked hydrogel compositions.

In some embodiments, the injectable shear-thinning compositions of the present disclosure flow upon application of a pressure greater than a yield stress of the injectable shear-thinning compositions.

Polymers for use in the injectable shear-thinning compositions of the present disclosure include uncharged polymers and charged polymers selected from anionic polymers and cationic polymers. Anionic polymers may contain one or more negatively charged functional groups such as carboxylate functional groups, sulfate functional groups, sulfonate functional groups, phosphonate functional groups, phosphate functional groups or any combination of the preceding functional groups. Cationic polymers may contain one or more positively charged functional groups such as amine functional groups, including primary amine functional groups, secondary amine functional groups, tertiary amine functional groups, and quaternary amine functional groups.

Polymers for use in the injectable shear-thinning compositions of the present disclosure include polymers that have a net-negative charge, have a net-positive charge, or are neutrally charged. Net-negative-charge polymers include polymers that contain negatively charged functional groups such as carboxylate functional groups, sulfate functional groups, sulfonate functional groups, phosphonate functional groups, phosphate functional groups or any combination of the preceding functional groups. Net-negative-charge polymers for use in the present disclosure typically have a net-negative charge at a pH of 8.0 or more. However, in some embodiments, net-negative-charge polymers for use in the present disclosure have a net-negative charge at a pH of 7.0, at a pH of 6.5, at a pH of 6.0, at a pH of 5.5, at a pH of 5.0, at a pH of 4.5, or even at a pH of 4.0. Net-positive charge polymers include polymers that contain positively charged functional groups such as amine functional groups, including primary amine functional groups, secondary amine functional groups, tertiary amine functional groups, and quaternary amine functional groups. Net-positive-charge polymers for use in the present disclosure typically have a net-positive charge at a pH of 6.0 in or less. However, in some embodiments, net-positive-charge polymers for use in the present disclosure have a net-positive charge at a pH of 6.5, at a pH of 7.0, at a pH of 7.5, at a pH of 8.0, at a pH of 8.5, at a pH of 9.0, at a pH of 9.5, or even at a pH of 10.0.

For example, polymers for use in the injectable shear-thinning compositions of the present disclosure include polysaccharides which may have a net-negative charge, may have a net-positive charge, or may be neutrally charged. Net-negative-charge polysaccharides include polysaccharides that contain negatively charged functional groups such as carboxylate functional groups, sulfate functional groups, sulfonate functional groups, phosphonate functional groups, phosphate functional groups or any combination of the preceding functional groups. Net-negative-charge polysaccharides for use in the present disclosure typically have a net-negative charge at a pH of 8.0 or more. In some embodiments, net-negative-charge polysaccharides for use in the present disclosure have a net-negative charge at a pH of 7.0, at a pH of 6.5, at a pH of 6.0, at a pH of 5.5, at a pH of 5.0, at a pH of 4.5, or even at a pH of 4.0.

Particular examples of polysaccharides having a net-negative charge include carboxylic-acid-containing polysaccharides that contain one or more uronic acid species, such as galacturonic acid, glucuronic acid and/or iduronic acid. Particular examples of carboxylic-acid-containing polysaccharides include pectin, agaropectin, carrageenan, gellan gum, gum arabic, guar gum, xanthan gum, alginic acid, hyaluronic acid, and carboxymethyl cellulose. In embodiments where the carboxylic-acid-containing polysaccharide is hyaluronic acid, the carboxylic-acid-containing polysaccharide may be non-animal stabilized hyaluronic acid. Another particular example of a polysaccharide having a net-negative charge is agar, which is mixture of polysaccharides containing agarose, which is a neutral polysaccharide, and agaropectin, which is a charged sulfated polysaccharide. Other particular examples of polysaccharides having a net-negative charge include carboxyalkyl celluloses such as carboxymethyl cellulose.

Net-positive charge polysaccharides include polysaccharides that contain positively charged functional groups such as amine functional groups, including primary amine functional groups, secondary amine functional groups, tertiary amine functional groups, and quaternary amine functional groups. Net-positive-charge polysaccharides for use in the present disclosure typically have a net-positive charge at a pH of 6.0 or less. In some embodiments, net-positive-charge polysaccharides for use in the present disclosure have a net-positive charge at a pH of 6.5, at a pH of 7.0, at a pH of 7.5, at a pH of 8.0, at a pH of 8.5, at a pH of 9.0, at a pH of 9.5, or even at a pH of 10.0. Particular examples of polysaccharides with a net-positive charge include chitosan and cationic starch.

Neutral polysaccharides include cellulose derivatives including alkyl celluloses such as methyl cellulose and ethyl cellulose and hydroxyalkyl celluloses such as hydroxyethyl cellulose and hydroxypropyl cellulose. Neutral polysaccharides further include starches such as corn starch, potato starch, and tapioca starch.

Polymers for use in the injectable shear-thinning compositions of the present disclosure also include animal-derived proteins such as collagen, including bovine collagen, porcine collagen and equine collagen, and gelatin, including porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

Polymers for use in the injectable shear-thinning compositions of the present disclosure further include non-animal-derived proteins including plant derived proteins and microbially derived proteins, including spider silk proteins, known as spidroins, which are expressed by bacteria and which demonstrate gelation due to increased temperature (37° C.) through α-helix to β-sheet conversion. His-NT2RepCT, a recombinant mini-spidroin that is composed of an N-terminal (NT), a short repeat region, a C-terminal (CT) and a His 6-tag for purification is as soluble as native spider silk proteins in aqueous buffers and possesses important features of native spider silk. See T. Arndt et. al., “Spidroin N-terminal domain forms amyloid-like fibril based hydrogels and provides a protein immobilization platform,” Nat Commun 13, 4695 (2022); T. Arndt et al. “Native-like Flow Properties of an Artificial Spider Silk Dope”, ACS Biomater Sci Eng. 2021 Feb. 8; (2): 462-471.

Polymers for use in the injectable shear-thinning compositions of the present disclosure include cationic polypeptides that contain cationic amino acids such as lysine, arginine and ornithine, including polylysine, polyarginine, polyornithine, and polypeptides containing two or more of lysine, arginine and ornithine.

Polymers for use in the injectable shear-thinning compositions of the present disclosure also include anionic polypeptides that contain anionic amino acids such as aspartic acid and glutamic acid, including polyaspartic acid, polyglutamic acid and polypeptides containing aspartic acid and glutamic acid.

Polymers for use in the injectable shear-thinning compositions of the present disclosure further include synthetic polymers having a net-negative charge such as polymethacrylic acid, polyacrylic acid, polyitaconic acid, or poly(2-acrylamido-2-methyl-1-propanesulfonic acid) (polyAMPS) and synthetic polymers having a net-positive charge such as poly(di-C₁-C₆-alkylaminoethanol acrylate) and poly(di-C₁-C₆-alkylaminoethanol methacrylate).

In some embodiments, the injectable shear-thinning compositions of the present disclosure include any two, three or more types of the polymers listed above. In some of these embodiments, the injectable shear-thinning compositions of the present disclosure include a net-positive charge polymer, for example, selected from those above, in combination with net-negative charge polymer, for example, selected from those above. In some of these embodiments, the injectable shear-thinning compositions of the present disclosure include gelatin and one or more types of the other polymers listed above.

In some embodiments, the injectable shear-thinning compositions of the present disclosure contain between 0.05 wt % and 60.0 wt % of one or more types of polymers. For example, injectable shear-thinning compositions of the present disclosure contain anywhere from 0.05 wt % to 0.10 wt % to 0.25 wt % to 0.5 wt % to 1 wt % to 2.5 wt % to 5 wt % to 10 wt % to 25 wt % to 50 wt % to 60 wt % (in other words ranging between any two of the preceding numerical values) of one or more types of polymers.

Microparticles for use in the injectable shear-thinning compositions of the present disclosure include metallic microparticles, metal oxide microparticles, carbon based microparticles, silicate microparticles, lipid microparticles and crosslinked charged polymer microparticles.

Examples of metallic microparticles for use in the injectable shear-thinning compositions of the present disclosure include transition metal microparticles such as titanium microparticles, tantalum microparticles, gold microparticles, silver microparticles, copper microparticles and iron microparticles, Group 2 metal microparticles such as magnesium microparticles, and alloy microparticles such as brass microparticles, steel microparticles, cobalt chrome microparticles and platinum chrome microparticles.

Examples of metal oxide microparticles for use in the injectable shear-thinning compositions of the present disclosure include iron oxide microparticles, titanium oxide microparticles, cobalt oxide microparticles (including cobalt (II), cobalt (III) and cobalt (II,III) oxide microparticles, gold (III) oxide microparticles, and tungsten oxide microparticles.

Examples of carbon-based microparticles for use in the injectable shear-thinning compositions of the present disclosure include activated carbon microparticles and graphite microparticles, including exfoliated graphite nanoparticles and exfoliated graphite platelets.

Examples of silicate microparticles for use in the injectable shear-thinning compositions of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL2I-based silicate nanoplatelets, and Laponite® D-based silicate nanoplatelets), Sumecton® SWN and Lucentite™ SWN, magnesium aluminum silicates such as Sumecton® SA, sodium magnesium silicates such as Optigel® SH and SUPLITE-MP, and fluoromica such as Somasif™ ME100, among others.

Examples of crosslinked charged polymers microparticles for use in the injectable shear-thinning compositions of the present disclosure include crosslinked poly(2-acrylamido-2-methyl-1-propanesulfonic acid) (polyAMPS), gelatin and chitosan.

Microparticles for use in the injectable shear-thinning compositions of the present disclosure may be provided in a variety of regular and irregular shapes and include spherical microparticles (e.g., regular spherical microparticles, oblate spherical microparticles, prolate spherical microparticles), regular geometric microparticles (e.g., cubic microparticles, tetrahedral microparticles, rhombohedral microparticles, hexagonal microparticles, octahedral microparticles, dodecahedral microparticles, icositetrahedral microparticles, prismatic microparticles, etc.), plate-shaped microparticles, and rod-shaped microparticles.

Microparticles for use in the injectable shear-thinning compositions of the present disclosure may have a size ranging from 10 nm or less to 600 μm or more in longest dimension (e.g., diameter for a sphere, length for a rod, greatest width for a platelet, etc.), for example, ranging anywhere from 10 nm to 25 nm to 50 nm to 100 nm to 250 nm to 500 nm to 1 μm to 2.5 μm to 5 μm to 10 μm to 25 μm to 50 μm to 100 μm to 250 μm to 600 μm.

Microparticles for use in the injectable shear-thinning compositions of the present disclosure include microparticles that have a neutral charge, microparticles that have a net positive charge, and microparticles that have a net negative charge,

In some embodiments, the microparticles inherently have a net positive charge or a net negative charge.

In some embodiments, the microparticles are surface modified such that they have a net positive charge or a net negative charge. For example, in order to provide the microparticles with a net positive charge, the microparticles may be surface modified to comprise positively charged functional groups such as amine functional groups, including primary amine functional groups, secondary amine functional groups, tertiary amine functional groups, quaternary amine functional groups, or any combination of the preceding functional groups. In order to provide the microparticles with a net negative charge, the microparticles may be surface modified to comprise negatively charged functional groups, such as carboxylate functional groups, sulfate functional groups, sulfonate functional groups, phosphonate functional groups, phosphate functional groups or any combination of the preceding functional groups.

Microparticles that have a net positive charge can be formed, for example, by covalently linking an organic molecule containing positively charged functional groups to the surfaces of the microparticles. Microparticles that have a net negative charge can be formed, for example, by covalently linking an organic molecule containing negatively charged functional groups to the surfaces of the microparticles.

Microparticles that have a net positive charge can also be formed, for example, by non-covalently linking an organic molecule containing positively charged functional groups to the surfaces of the microparticle. Similarly, microparticles that have a net negative charge can be formed, for example, by non-covalently linking an organic molecule containing negatively charged functional groups to the surfaces of the microparticles.

For example, microparticles that have a net positive charge can be formed by adsorbing a cationic surfactant to the surfaces of the microparticles. Cationic surfactants include, for example, cetyltrimethylammonium bromide or “CTAB” (e.g., cetrimide), cetyltrimethylammonium chloride (CTAC), benzalkonium chloride, benzethonium chloride, didodecyldimethyl ammonium bromide (DDAB), dioleoyl-3-trimethylammonium-propane (DOTAP), benzalkonium chloride, hexadecyl trimethyl ammonium chloride, dimethyidodecylaminopropane, N-cetyl-N-ethyl morpholinium ethosulfate, lauryltrimethylammonium chloride, stearyltrimethylammonium chloride, and cetylpyridinium chloride, among others.

As another example, microparticles that have a net negative charge can be formed by adsorbing an anionic surfactant to the surfaces of the microparticles. Anionic surfactants include, for example, sodium dodecyl sulfate (SDS), sodium lauryl sulfate (SLS), disulfosuccinate (DSS), sulphated fatty alcohols, sodium and potassium salts of fatty acids, glycerol esters of fatty acids, polyoxyl stearate, polyyoxyethylene lauryl ether, sorbitan sesquioleate, and triethanolamine, among others.

In some embodiments, the injectable shear-thinning compositions of the present disclosure include any two, three or more types of microparticles, for example, selected from the microparticles listed above. In some of these embodiments, the injectable shear-thinning compositions of the present disclosure include silicate microparticles, for example, lithium magnesium sodium silicates such as Laponite® silicate nanoplatelets, and one or more types of the other microparticles listed above. In some of these embodiments, the injectable shear-thinning compositions of the present disclosure include metallic microparticles, for example, tantalum microparticles, and one or more types of the other microparticles listed above.

In some embodiments, the injectable shear-thinning compositions of the present disclosure contain between 1 wt % and 50 wt % (e.g., ranging anywhere from 1 wt % to 2.5 wt % to 5 wt % to 10 wt % to 25 wt % to 50 wt %) of one or more types microparticles.

The water in the injectable shear-thinning compositions of the present disclosure may be provided in the form of ultrapure water, water for injection, saline, phosphate buffered saline, or high-ion-content water.

In some embodiments, the injectable shear-thinning compositions of the present disclosure contain between 5 wt % and 90 wt % (e.g., ranging anywhere from 5 wt % to 10 wt % to 25 wt % to 50 wt % to 75 wt % to 90 wt %) of water.

The injectable shear-thinning compositions of the present disclosure may be formed using a variety of methods. The one or more types of polymers, the one or more types of microparticles and the water may be mixed in any order. For example, the one or more types polymers and the one or more types of microparticles may be first mixed and combined with the water. As another example, the one or more types of polymers and the water may be first mixed and then combined with the one or more types of microparticles. As another example, the one or more types of microparticles and the water may be first mixed and then combined with the one or more types of polymers. As yet another example, the one or more types of microparticles, the one or more types of polymers and the water may be simultaneously mixed. Mixing may be performed by any suitable mixing technique, including, for example, centrifugal mixing, manual mixing, high shear dispersing, vacuum mixing, vortexing, and/or syringe-to-syringe mixing.

The injectable shear-thinning compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

In various embodiments, the injectable shear-thinning compositions of the present disclosure contain one or more agents in addition to one or more types of polymers, the one or more types of microparticles and the water. Examples of such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable shear-thinning compositions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imagable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

In various embodiments, the injectable shear-thinning compositions in accordance with the present disclosure have a radiopacity that is greater than 100 Hounsfield units (HU), beneficially ranging anywhere from 100 HU to 250 HU to 500 HU to 750 HU to 1000 HU or more. Radiopacity may be provided in a variety of ways. For example, one or more types of microparticles in the injectable shear-thinning compositions may provide radiopacity, one or more types of polymers (e.g., iodinated polymers) in the injectable shear-thinning compositions may provide radiopacity and/or one or more types of ionic and/or non-ionic radiocontrast agents (such as those listed above, among others) may provide radiopacity.

The injectable shear-thinning compositions of the present disclosure may be stored and transported in a sterile form. The injectable shear-thinning compositions may be shipped, for example, in a syringe, catheter, vial, ampoule, or other container.

In various embodiments, kits are provided, which may include one or more containers of shear-thinning compositions as described herein as well other components. For example, the kits may include one or more delivery devices for delivering the injectable shear-thinning compositions to a subject such as syringes, catheters or tubing sets. In some embodiments, the kits may comprise a shear-thinning composition as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g. contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable shear-thinning compositions described herein can be administered by a variety of routes, depending upon the desired medical outcome. In some embodiments, the administering comprises injecting the injectable shear-thinning composition. In some embodiments, the injectable shear-thinning compositions are administered by parenteral administration. Parenteral administration includes intravenous, intraarterial, subcutaneous, intraperitoneal, or intramuscular injection or infusion. In some embodiments, the administering comprises an image guided procedure where computed tomography, fluoroscopy or ultrasound imaging is used to deliver the composition. In some embodiments, the administering comprises injecting the injectable shear-thinning composition into the vascular system of a subject. In some embodiments, the administering comprises injecting the injectable shear-thinning composition into a cancer of the subject or the vasculature supplying a cancer of the subject. In some embodiments, the administering is performed using a catheter or a syringe.

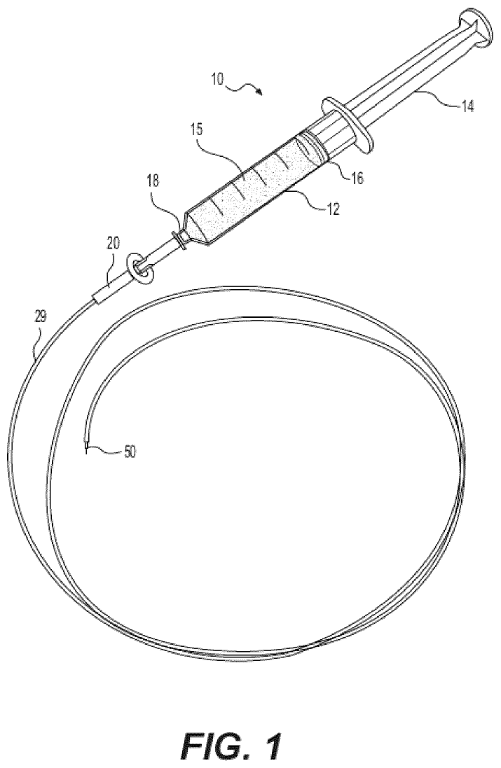

FIG. 1 illustrates an exemplary syringe 10 providing a reservoir for a shear-thinning composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a shear-thinning composition 15 for injection through the needle 50.

The injectable shear-thinning compositions described herein can be administered to patients for achieving a number of medical outcomes.

The injectable shear-thinning compositions described herein can be visualized (e.g., within a mammal) using any appropriate method during and/or after administration. For example, imaging techniques such as ultrasound, computed tomography, magnetic resonance imaging, and/or fluoroscopy can be used to visualize the injectable shear-thinning compositions provided herein.

The injectable shear-thinning compositions can be injected to provide spacing between tissues, the injectable shear-thinning compositions can be injected (e.g., in the form of blebs) to provide fiducial markers, the injectable shear-thinning compositions can be injected for tissue augmentation or regeneration, the injectable shear-thinning compositions can be injected as a filler or replacement for soft tissue, the injectable shear-thinning compositions can be injected to provide mechanical support for compromised tissue, the injectable shear-thinning compositions can be injected as a scaffold, and/or the injectable shear-thinning compositions can be injected as a carrier of therapeutic agents in the treatment of diseases and cancers and the repair and regeneration of tissue, among other uses.

The injectable shear-thinning compositions of the present disclosure may be used in a variety of medical procedures, including the following, among others: a procedure to implant a fiducial marker comprising the radiopaque particles, a procedure to implant a tissue regeneration scaffold comprising the radiopaque particles, a procedure to implant a tissue support comprising the radiopaque particles, a procedure to implant a tissue bulking agent comprising the radiopaque particles, a procedure to implant a therapeutic-agent-containing depot comprising the radiopaque particles, a tissue augmentation procedure comprising implanting the radiopaque particles, a procedure to embolize tissue, including benign tumors, malignant tumors and other abnormal tissue, a procedure to control bleeding, a procedure to introduce the radiopaque particles between a first tissue and a second tissue to space the first tissue from the second tissue.

The injectable shear-thinning compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable shear-thinning compositions may be injected for the permanent or temporary occlusion of blood vessels, and thus may be useful for managing various diseases and conditions. For example, the injectable shear-thinning compositions may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Shear-thinning compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Shear-thinning compositions in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. An injectable shear-thinning composition comprising (a) one or more types of polymers, (b) one or more types of microparticles and (c) water.

2. The injectable shear-thinning composition of claim 1, wherein the injectable shear-thinning composition is a physically crosslinked hydrogel composition.

3. The injectable shear-thinning composition of claim 1, wherein the one or more types of polymers comprise a polymer having a net-negative charge and/or a polymer having a net-positive-charge.

4. The injectable shear-thinning composition of claim 1, wherein the one or more types of polymers are selected from polysaccharides, proteins, polypeptides, and synthetic polymers.

5. The injectable shear-thinning composition of claim 1, wherein the one or more types of polymers are selected from non-animal-derived proteins and non-animal-derived polysaccharides.

6. The injectable shear-thinning composition of claim 1, wherein the one or more types of microparticles are selected from metallic microparticles, metal oxide microparticles, carbon based microparticles, silicate microparticles, lipid microparticles and crosslinked charged polymer microparticles.

7. The injectable shear-thinning composition of claim 1, wherein the one or more types of microparticles comprise microparticles having a net-negative charge and/or microparticles having a net-positive charge.

8. The injectable shear-thinning composition of claim 1, wherein the one or more types of microparticles have a size ranging from 10 nm to 50 μm in longest dimension.

9. The injectable shear-thinning composition of claim 1, wherein the one or more types of polymers comprise a polymer having a first net charge and wherein the one or more types of microparticles comprise microparticles having a second net charge that is opposite in sign to the first net charge.

10. The injectable shear-thinning composition of claim 1, wherein the injectable shear-thinning composition further comprises one or more additional agents selected from therapeutic agents, imaging agents, colorants, tonicity adjusting agents, suspension agents, wetting agents, and pH adjusting agents.

11. The injectable shear-thinning composition of claim 1, wherein the injectable shear-thinning composition is a sterile composition.

12. The injectable shear-thinning composition of claim 1, wherein the injectable shear-thinning composition is provided in a container.

13. The injectable shear-thinning composition of claim 1, wherein the injectable shear-thinning composition is provided in a preloaded syringe.

14. A kit comprising one or more containers that contain (a) an injectable shear-thinning composition comprising one or more types of polymers, one or more types of microparticles and water and (b) a delivery device.

15. The kit of claim 14, wherein the delivery device comprises a syringe, a needle and optionally, a catheter.

16. A medical procedure comprising administering to a subject an injectable shear-thinning composition comprising one or more types of polymers, one or more types of microparticles and water.

17. The medical procedure of claim 16, wherein the method comprises injecting the injectable shear-thinning composition into the subject.

18. The medical procedure of claim 16, wherein the administering comprises parenteral administration.

19. The medical procedure of claim 16, wherein the administering is performed using a catheter or a syringe.

20. The medical procedure of claim 16, wherein the administering is performed under image guidance.
